(19) United States
(12) Patent Application Publication
(10) Pub. No.: US 2024/0158816 A1
(43) Pub. Date: May 16, 2024

(54) METHODS AND COMPOSITIONS FOR THE PRODUCTION AND DEHYDRATION OF ISOPRENOL INTO ISOPRENE

(71) Applicant: THE REGENTS OF THE UNIVERSITY OF CALIFORNIA, Oakland, CA (US)

(72) Inventors: Eric SUNDSTROM, San Mateo, CA (US); Carolina ARAUJO BARCELOS, Emeryville, CA (US); Taek Soon LEE, Berkeley, CA (US)

(21) Appl. No.: 18/494,656

(22) Filed: Oct. 25, 2023

Related U.S. Application Data
(60) Provisional application No. 63/381,175, filed on Oct. 27, 2022.

(51) Int. Cl. C12P 5/00; C07C 1/24; C10L 1/04; C12N 9/88

(52) U.S. Cl. CPC C12P 5/002; C07C 1/24; C10L 1/04; C12N 9/88; C12P 5/007; C12Y 401/01; C10L 2200/043; C10L 2200/0446

(57) ABSTRACT

The present invention provides for a method for producing isoprene and/or 1,4-dimethylcyclooctane (DMCO), the method comprising: (a) producing isoprenol (3-methyl-3-buten-1-ol) biologically; (b) recovering the isoprenol via gas stripping; (c) dehydrating the isoprenol into isoprene; and, (d) optionally converting the isoprene into DMCO.

### CROSS-REFERENCE TO RELATED APPLICATIONS

This application claims priority to U.S. Provisional Patent Application Ser. No. 63/381,175, filed on Oct. 27, 2022, which is hereby incorporated by reference.

### REFERENCE TO SEQUENCE LISTING

The instant application contains a Sequence Listing which has been submitted electronically in XML format and is hereby incorporated by reference in its entirety. Said XML copy, created on Oct. 25, 2023, is named “2021-096-03 Sequence Listing.xml” and is 4 bytes in size.

### STATEMENT OF GOVERNMENTAL SUPPORT

The invention described and claimed herein was made utilizing funds supplied by the U.S. Department of Energy under Contract No. DE-ACO2-05CH11231. The government has certain rights in this invention.

### FIELD OF THE INVENTION

The present invention is in the field of production of producing isoprene.

### BACKGROUND OF THE INVENTION

Current methods of production of 1,4-dimethylcyclooctane (DMCO) are entirely through chemical synthesis. Also, the existing production of isoprene relies on petrochemical feedstocks. The direct biological production of isoprene results in volatilization of the product into a high-oxygen gas stream, creating a substantial flammability hazard, particularly during product recovery. In addition, this capture of isoprene from such a stream is extremely difficult, necessitating high cost adsorbents and/or condensation at cryogenic temperatures. By contrast, isoprenol does not form flammable vapors at fermentation temperatures, and separation from the off-gas stream is relatively simple. A procedure is needed whereby this process is coupled directly to the dehydration of aqueous isoprenol, so that any need for distillation and purification of the isoprenol intermediate is eliminated, while simultaneously regenerating the capture solvent.

Isoprene can be catalytically upgraded through dehydration, dimerization and hydrogenation into 1,6-dimethyl-1,5-cyclooctadiene (DMCOD) and 1,4-dimethylcyclooctane (DMCO) (Rosenkoetter et al., “[4+4]-cycloaddition of isoprene for the production of high-performance bio-based jet fuel,” Green Chemistry 20 (2019); Baral et al., “Production Cost and Carbon Footprint of Biomass-Derived Dimethylcyclooctane as a High-Performance Jet Fuel Blendstock,” ACS Sustainable Chem. Eng. 2021, 9, 11872-11882).

### SUMMARY OF THE INVENTION

The present invention provides for a method for producing isoprene and/or 1,4-dimethylcyclooctane (DMCO), the method comprising: (a) producing isoprenol (3-methyl-3-buten-1-ol) biologically; (b) recovering the isoprenol via gas stripping; (c) dehydrating the isoprenol into isoprene; and, (d) optionally converting the isoprene into DMCO.

In some embodiments, the method comprises: (1) biological production of isoprenol, (2) recovery of isoprenol via gas stripping into organic solvent (octanol, etc), (3) extraction of isoprenol into water via liquid-liquid extraction, (4) dehydration of isoprenol into isoprene, and (5) optionally chemical conversion of isoprene into DMCO.

FIG. 7 shows an embodiment of the method of the invention, the method comprising: step 10 is producing isoprenoi biologically, step 20 is recovering isoprenol via gas stripping, step 30 is dehydrating isoprenol into isoprene, and step 40 is converting isoprene into DMCO. The converting of isoprene into DMCO is a chemical conversion of isoprene into DMCO. In some embodiments, the gas stripping comprises gas stripping into an organic solvent, such as octanol, 2-ethyl-1,3-hexanediol, or oleyl alcohol. In some embodiments, the gas stripping comprises gas stripping into an aqueous phase, such as water. In some embodiments, the solvent is of a low temperature equal to or lower than about 15° C., 10° C., 5° C., 4° C., 3° C., 2° C., 1° C., or 0° C. In some embodiments, the solvent comprises an organic phase and/or an aqueous phase.

Both isoprenol and isoprene are potentially high volume biochemical molecules accessible via high flux metabolic pathways. Isoprenol is a highly promising target molecule for biological production, both due to its value as a high octane gasoline blendstock, and due to its relatively facile conversion to the platform molecule isoprene. Isoprene is a high volume commodity chemical with extensive use in the polymer industry, and it is readily dimerized and trimerized to drop in jet and diesel fuels. Biological production of isoprenol is limited by product toxicity, and biological production of isoprene is limited by flammability and off-gas capture challenges. The biological production of isoprenol is hindered by its toxicity to the production host at concentrations above about 10 g/L. Direct biological production of isoprene is possible, however the extreme volatility of isoprene and the necessary aerobic fermentation conditions create substantial challenges to its off-gas capture and due to its flammability.

In some embodiments, aeration used for oxygen supply to fermentation is used to strip isoprenol to fermentor off-gas, removing it from the fermentation medium. Isoprenol is then continuously captured from the off-gas stream via gas stripping in chilled solvent or water, and the resulting aqueous isoprenol is continuously dehydrated to isoprene and captured as a high purity gas. Continuous dehydration of isoprenol to isoprene then allows recycling of purified water to the off-gas capture system. In this way, isoprenol toxicity is overcome via gas stripping, and isoprenol can be capture and converted to isoprene with high efficiency and no required distillation.

Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. The chemical structures of prenol (top) and isoprenol (bottom).

FIG. 2. A set-up for liquid-liquid extractive fermentation.

FIG. 3. A set-up for off-gas stripping in organic phase.

FIG. 4. Measured results for an integrated volatile capture and conversion experiment.

FIG. 5. Isoprenol retention over time using different solvents for capture.

FIG. 6. A scheme for converting isoprene into DMCO.

FIG. 7. An embodiment of the invention for a method for producing isoprene and/or DMCO.

FIG. 8. An embodiment of the device or apparatus for the production and gas-stripping of isoprenol useful for the present invention.

FIG. 9. Amino acid sequence comparison between Saccharomyces cerevisiae PMD (Seq_1, SEQ ID NO:1) and S. epidermis PMD (Seq_2, SEQ ID NO:2).

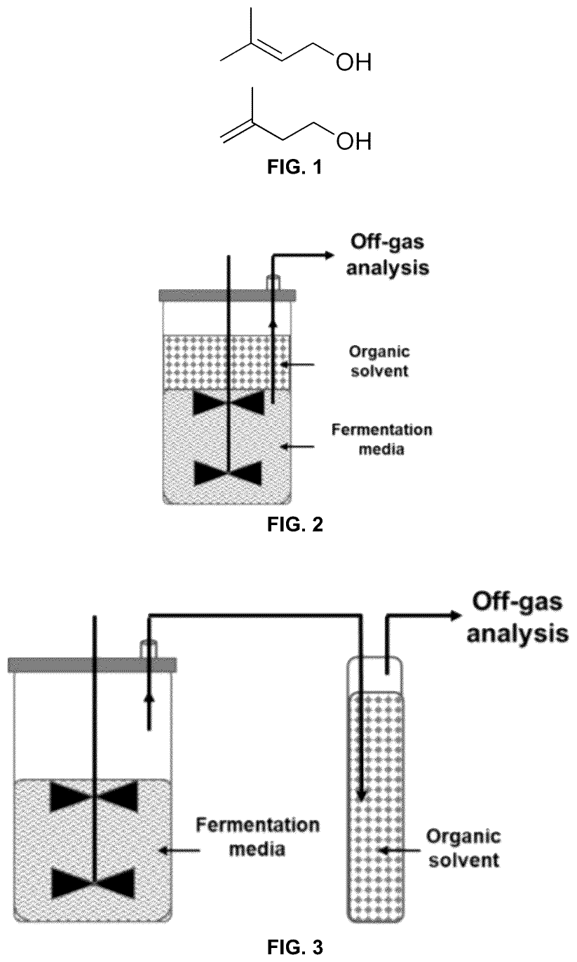

### DETAILED DESCRIPTION OF THE INVENTION

Before the invention is described in detail, it is to be understood that, unless otherwise indicated, this invention is not limited to particular sequences, expression vectors, enzymes, host cells, microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes up to 10% less and 10% more than the value referred to.

The terms “host cell” and “host microorganism” are used interchangeably herein to refer to a living biological cell, such as a microorganism, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids comprises two or more subsequences that are not found in the same relationship to each other in nature. The term “heterologous” as used herein refers to a structure or molecule wherein at least one of the following is true: (a) the structure or molecule is foreign to (i.e., not naturally found in) a given host cell; or (b) the structure or molecule may be naturally found in a given host cell, but in an unnatural (e.g., greater than expected) amount. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host cell, wherein the expression vector contains a nucleic acid sequence coding for an enzyme that is not normally found in a host cell. With reference to the host cell's genome, then, the nucleic acid sequence that codes for the enzyme is heterologous.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host cell or cell. Only when the sequence of nucleic acids becomes stably replicated by the cell does the host cell or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host cell, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; intemucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., arninoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

### Producing Isoprenol Biologically

In some embodiments, the producing isoprenol biologically step comprises one or more, or all, of the following steps: providing for a host cell capable of producing isoprenol, and culturing the host cell under a condition wherein isoprenol is produced. In some embodiments, the host cell is naturally capable of producing isoprenol, or is genetically modified to produce isoprenol (wherein the host cell in its unmodified form does not produce isoprenol), or is genetically modified to produce more isoprenol than in its unmodified form (wherein the host cell in its unmodified form produces isoprenol albeit less than in its modified form).

In some embodiment, the host cell capable of producing isoprenol is a genetically modified host cell capable of producing more isoprenol than the unmodified host cell. In some embodiment, the host cell capable of producing isoprenol is a genetically modified host cell capable of producing isoprenol, comprising (a) an increased expression of phosphomevalonate decarboxylase (PMD), or a homologous enzyme thereof, (b) an increased expression of a phosphatase, or a homologous enzyme thereof, capable of converting isopentenol into isoprenol, (c) optionally the genetically modified host cell does not express, or has a decreased expression of one or more of NudB, PMK, and/or PMD, or a homologous enzyme thereof, and (d) optionally one or more further enzymes capable of converting isoprenol into a third compound, such as isoprene. In some embodiments, the decreased expression is a disruption of the promoter or knock out of the gene encoding the enzyme.

In some embodiments, the genetically modified host cell further comprises an increased expression of one or more of AtoB, hydroxymethylglutaryl-CoA synthase (HMGS), hydroxymethylglutaryl-CoA reductase (HMGR), and/or MK, or a homologous enzyme thereof.

In some embodiments, one or more of the described expressed enzymes, such as PMD, phosphatase, AtoB, HMGS, HMGR, and/or MK, or a homologous enzyme thereof, are encoded on one or more nucleotide sequences which are in one or more nucleic acids which are transformed into the genetically modified host cell, or host cell prior to genetic modification. In some embodiments, the nucleotide sequences encoding the one or more enzymes are operatively linked to one or more promoters capable of transcription in the genetically modified host cell. In some embodiments, each nucleic acid of the one or more nucleic acids is a vector capable of stable introduction into and/or maintenance in the host cell.

In some embodiment, the culturing step is culturing the genetically modified host cell under a condition wherein PMD and/or phosphatase are expressed. In some embodiments, the culturing step further comprises expressing AtoB, HMGS, HMGR, and/or MK. In some embodiments, the culturing step is under an anaerobic or microaerobic condition.

In some embodiments, one or more of the enzymes, including PMD, phosphatase, AtoB, HMGS, HMGR, and MK, is an engineered enzyme, or homologous, mutant or variant enzymes having the same enzymatic activity, with an amino acid sequence having equal to or more than 70%, 80%, 90%, 95%, or 99% identity to the amino acid sequence of the corresponding wild-type enzyme, such as the specific enzymes described in U.S. Pat. Nos. 10,273,506 and 11,001,838 (both of which are incorporated by reference).

In some embodiments, one or more of the enzymes, including PMD, phosphatase, AtoB, HMGS, HMGR, and MK, is heterologous to the host cell.

In some embodiments, the genetically modified host cell is capable of producing one or more compounds in titers or yields equal to or more than the titers or yields described herein.

In some embodiments, the genetically modified host cell capable of producing isoprenol, comprising (a) an increased expression of phosphomevalonate decarboxylase (PMD), wherein the PMD has an amino acid sequence having at least 70% identity with SEQ ID NO:1 or SEQ ID NO:2, and (i) amino acid residue at position 74 is histidine, (ii) amino acid residue at position 145 is phenylalanine, or (iii) amino acid residue at position 74 is histidine and amino acid residue at position 145 is phenylalanine, (b) an increased expression of a phosphatase capable of converting isopentenol into isoprenol, (c) optionally the genetically modified host cell does not express, or has a decreased expression of one or more of dihydroneopterin triphosphate diphosphate (NudB), phosphomevalonate kinase (PMK), and/or PMD, and (d) optionally one or more further enzymes capable of converting isoprenol into a third compound. In some embodiments, the host cell is a bacterial or fungal cell.

In some embodiments, the decreased expression is a disruption of the promoter or knock out of the gene encoding NudB, PMK, and/or PMD. In some embodiments, the third compound 15 isoprene.

In some embodiments, the genetically modified host cell further comprises an increased expression of one or more of acetyl-CoA acetyltransferase (AtoB), hydroxymethylglutaryl-CoA synthase (HMGS), hydroxymethylglutaryl-CoA reductase (HMGR), and/or mevalonate kinase (MK).

In some embodiments, the PMD is encoded on a nucleotide sequence which is in nucleic acids which is transformed into the genetically modified host cell, or host cell prior to genetic modification.

In some embodiments, one or more of the PMD, phosphatase, AtoB, HMGS, HMGR, and MK, are encoded on one or more nucleotide sequences which are in one or more nucleic acids which are transformed into the genetically modified host cell, or host cell prior to genetic modification.

In some embodiments, the step for producing isoprenol comprises: (a) providing a genetically modified host cell of the invention, (b) culturing the genetically modified host cell under a condition wherein phosphomevalonate decarboxylase (PMD) and/or phosphatase are expressed, and (c) optionally recovering the isopentenol and/or 3-methyl-3-butenol and/or the third compound. In some embodiments, the (b) culturing step further comprises expressing acetyl-CoA acetyltransferase (AtoB), hydroxymethylglutaryl-CoA synthase (HMGS), hydroxymethylglutaryl-CoA reductase (HMGR), and/or mevalonate kinase (MK). In some embodiments, the (b) culturing step is under an anaerobic or microaerobic condition.

In some embodiments, the genetically modified host cell is a host cell or microorganism, disclosed by U.S. Pat. Nos. 9,752,163; 9,879,286; 10,273,506; and, 10,814,724; and, PCT International Patent Application Nos. PCT/EP2009/067784 and PCT/US2021/040757; and by Zheng, et al. “Metabolic engineering of Escherichia coli for high-specificity production of isoprenol and prenol as next generation of biofuels,” Biotechnology for Biofuels 6:57 (2013); George, et al. “Metabolic engineering for the high-yield production of isoprenoid-based C₅ alcohols in E. coli,” Scientific Reports 5:11128 (2015); Wang, et al. “Tolerance Characterization and Isoprenol Production of Adapted Escherichia coli in the Presence of Ionic Liquids,” ACS Sustainable Chem. Eng. 7(1):1457-1463 (2018); and, Kim et al. “Engineerin Saccharomyces cerevisiae for isoprernol production” Metabolic Engineering 64:154-166 (2021) (whereby all are incorporated by reference in their entireties).

In some embodiments, the PMD comprises the following amino acid residues: (a) E at position 73, S at position 108, N at position 110, A at position 119, S at position 120, S at position 121, A at position 122, S at position 155, R at position 158, S at position 208, and/or D at position 302 corresponding to SEQ ID NO:1; or (b) Eat position 71, S at position 94, N at position 96, A at position 105, S at position 106, S at position 107, A at position 108, S at position 141, R at position 144, S at position 192, and/or D at position 283 corresponding to SEQ ID NO:2.

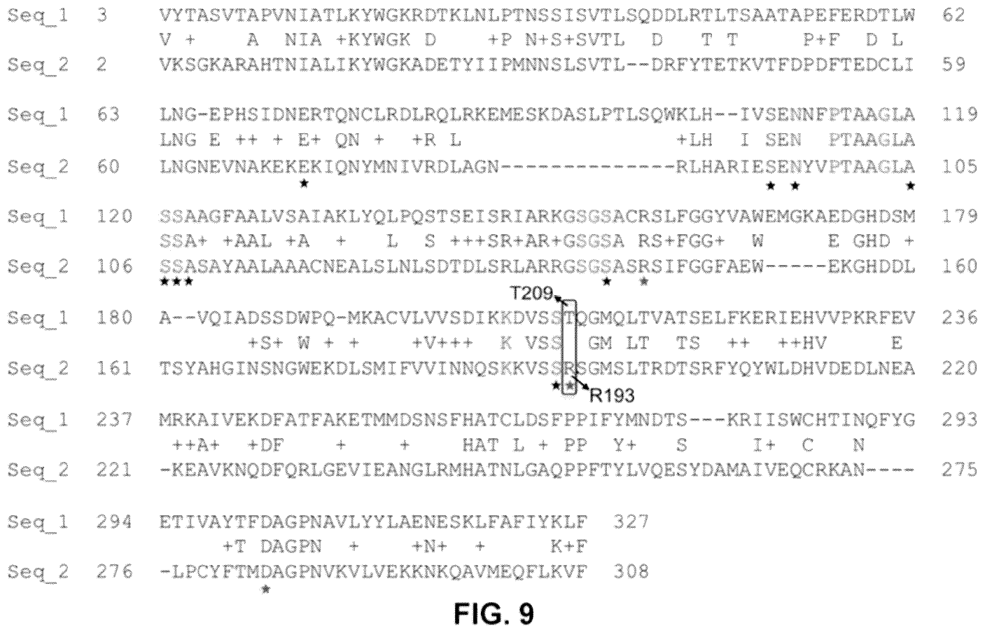

The PMD is any suitable PMD, such as any PMD with an amino acid sequence substantially identical to the amino acid sequences of SEQ ID NO: 1 or 2. The substantially identical PMD comprises one or more, or all, of the conserved residues are identified in FIG. 9, including but not limited to one or more, or all, of the conserved residues indicated by a star.

The amino acid sequence of Saccharomyces cerevisiae PMD (SEQ ID NO:1) is as follows:

```
                10 20 30 40
MTVYTASVTA PVNIATLKYW GKRDTKLNLP TNSSISVTLS
                50 60 70 80
QDDLRTLTSA ATAPEFERDT LWLNGEPHSI DNERTQNCLR
                90 100 110 120
DLRQLRKEME SKDASLPTLS QWKLHIVSEN NFPTAAGLAS
              130 140 150 160
SAAGFAALVS AIAKLYQLPQ STSEISRIAR KGSGSACRSL
              170 180 190 200
FGGYVAWEMG KAEDGHDSMA VQIADSSDWP QMKACVLVVS
              210 220 230 240
DIKKDVSSTQ GMQLTVATSE LFKERIEHVV PKRFEVMRKA
              250 260 270 280
IVEKDFATFA KETMMDSNSF HATCLDSFPP IFYMNDTSKR
              290 300 310 320
IISWCHTINQ FYGETIVAYT FDAGPNAVLY YLAENESKLF
              330 340 350 360
AFIYKLFGSV PGWDKKFTTE QLEAFNHQFE SSNFTARELD
              370 380 390
LELQKDVARV ILTQVGSGPQ ETNESLIDAK TGLPKE
```

The amino acid sequence of Saccharomyces epidermis PMD (SEQ ID NO:2) is as follows:

```
Met Val Lys Ser Gly Lys Ala Arg Ala His Thr Asn
1 5 10
Ile Ala Leu Ile Lys Tyr Trp Gly Lys Ala Asp Glu
                15 20
Thr Tyr Ile Ile Pro Met Asn Asn Ser Leu Ser Val
25 30 35
Thr Leu Asp Arg Phe Tyr Thr Glu Thr Lys Val Thr
                        40 45
Phe Asp Pro Asp Phe Thr Glu Asp Cys Leu Ile Leu
        50 55 60
Asn Gly Asn Glu Val Asn Ala Lys Glu Lys Glu Lys
                                65 70
Ile Gln Asn Tyr Met Asn Ile Val Arg Asp Leu Ala
                75 80
Gly Asn Arg Leu His Ala Arg Ile Glu Ser Glu Asn
85 90 95
Tyr Val Pro Thr Ala Ala Gly Leu Ala Ser Ser Ala
                        100 105
Ser Ala Tyr Ala Ala Leu Ala Ala Ala Cys Asn Glu
        110 115 120
Ala Leu Ser Leu Asn Leu Ser Asp Thr Asp Leu Ser
                                125 130
Arg Leu Ala Arg Arg Gly Ser Gly Ser Ala Ser Arg
                135 140
Ser Ile Phe Gly Gly Phe Ala Glu Trp Glu Lys Gly
145 150 155
His Asp Asp Leu Thr Ser Tyr Ala His Gly Ile Asn
                        160 165
Ser Asn Gly Trp Glu Lys Asp Leu Ser Met Ile Phe
        170 175 180
Val Val Ile Asn Asn Gln Ser Lys Lys Val Ser Ser
                                185 190
Arg Ser Gly Met Ser Leu Thr Arg Asp Thr Ser Arg
                195 200
Phe Tyr Gln Tyr Trp Leu Asp His Val Asp Glu Asp
205 210 215
Leu Asn Glu Ala Lys Glu Ala Val Lys Asn Gln Asp
                        220 225
Phe Gln Arg Leu Gly Glu Val Ile Glu Ala Asn Gly
        230 235 240
Leu Arg Met His Ala Thr Asn Leu Gly Ala Gln Pro
                                245 250
Pro Phe Thr Tyr Leu Val Gln Glu Ser Tyr Asp Ala
                255 260
Met Ala Ile Val Glu Gln Cys Arg Lys Ala Asn Leu
265 270 275
Pro Cys Tyr Phe Thr Met Asp Ala Gly Pro Asn Val
                        280 285
Lys Val Leu Val Glu Lys Lys Asn Lys Gln Ala Val
        290 295 300
Met Glu Gln Phe Leu Lys Val Phe Asp Glu Ser Lys
                                305 310
Ile Ile Ala Ser Asp Ile Ile Ser Ser Gly Val Glu
                315 320
Ile Ile Lys
325
```

Enzymes, and Nucleic Acids Encoding thereof

A homologous enzyme is an enzyme that has a polypeptide sequence that is at least 70%, 75%, 80%, 85%, 90%, 95% or 99% identical to any one of the enzymes described in this specification or in an incorporated reference. The homologous enzyme retains amino acids residues that are recognized as conserved for the enzyme. The homologous enzyme may have non-conserved amino acid residues replaced or found to be of a different amino acid, or amino acid(s) inserted or deleted, but which does not affect or has insignificant effect on the enzymatic activity of the homologous enzyme. The homologous enzyme has an enzymatic activity that is identical or essentially identical to the enzymatic activity any one of the enzymes described in this specification or in an incorporated reference. The homologous enzyme may be found in nature or be an engineered mutant thereof.

The nucleic acid constructs of the present invention comprise nucleic acid sequences encoding one or more of the subject enzymes. The nucleic acid of the subject enzymes are operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, Xhol, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host cell simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, NY.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host cell. Also, microinjection of the nucleic acid sequencers) provides the ability to transfect host cell. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

When the host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of the isoprenol ensured. When added, any intermediate is present in an excess amount in the culture medium.

An elevated level of producing isoprenol is more than 40 μg/10 mL (weight cell volume). In some embodiments, the elevated level of producing isoprenol is the genetically modified host cell capable of producing equal to or more than about 1 mg, 5 mg, 10 mg, 20 mg, 30 mg, 40 mg, 50 mg, 60 mg, 70 mg, 80 mg, 90 mg, 100 mg, 110 mg, 120 mg, or 130 mg per liter medium. In some embodiments, the medium is a complex medium, such as yeast extract peptone dextrose (YPD) medium. In some embodiments, the medium is minimal medium, such as Delft medium.

In some embodiments, the genetically modified host cell is engineered to overexpress EfmvaE and Efmvas from Enterococcus faecalis, and ERG8_{sc}, ERG12_{sc}, and ERG19_{sc} from S. cerevisiae, and NudB, or any homologous enzyme thereof. The increased expression of each enzyme can be from the expression from one or more stably introduced genes encoding the enzyme to the host cell. The introduced gene can be stable integrated into the host cell genome or reside on one or more plasmids, such as a high copy number plasmid, in the host cell.

In some embodiments, the genetically modified host cell is engineered to be knocked out for an endogenous phosphomevalonate kinase or PMK (such as ERGS), a cytosolic enzyme that acts in the biosynthesis of isoprenoids and sterols, and/or an endogenous choline kinase, and optionally to have an increased expression of a phosphatase, such as increased expression of an endogenous phosphatase, or one or more phosphatases expressed from one or more stably introduced genes encoding a phosphatase native or heterologous to the host cell. The stably introduced phosphatase genes can encode one or more of the following phosphatases: PHO5, acid phosphatase from S. cerevisiae; PHO3, acid phosphatase from S. cerevisiae; LPP1, lipid phosphate phosphatase from S. cerevisiae; PYP, sugar alcohol phosphatase from S. cerevisiae; GLC7, serine/threonine phosphatase from S. cerevisiae; PAH1, phosphatidate phosphatase from S. cerevisiae; DPP1, diacylglycerol phosphate phosphatase from S. cerevisiae; PHO13, alkaline phosphatase from S. cerevisiae; GPP1, glycerol-3-phosphatase from S. cerevisiae; GPP2, glycerol-3-phosphatase from S. cerevisiae; APHA, aminoglycoside-3-phosphotransferase from E. coli; AGP, glucose-1-phosphatase from E. coli; YQAB, fructose-1-phosphate from E. coli; PHO8, alkaline phosphatase from S. cerevisiae; PHOA, alkaline phosphatase from E. coli, or any homologous enzyme thereof. In some embodiments, the nucleotide sequence encoding the phosphatase is codon optimized for the genetically modified host cell. The phosphatase can be one that is naturally occurring or synthetic.

The present invention provides for a method for constructing genetically modified host cell of the present invention comprising: (a) introducing one or more nucleic acid comprising open reading frames (ORF) encoding the enzymes described herein wherein each is operatively linked to a promoter capable of transcribing each ORF to which it is operatively linked, and/or (b) knocking out one or more of the enzymes described herein such that the modified host cell does not express the one or more knocked out enzymes.

The present invention provides for a method for producing an isoprenol comprising: (a) providing the genetically modified host cell of the present invention, or a culture thereof, (b) culturing or growing the genetically modified host cell to produce the isoprenol, (c) optionally extracting or separating the isoprenol from the culture, and (d) optionally introducing a fuel additive to the extracted or separated the isoprenol. In some embodiments, the step of extracting or separating the isoprenol is concurrent or subsequent to the culturing or growing step.

### Host Cells

The genetically modified host cell can be any prokaryotic or eukaryotic cell, with any genetic modifications, capable of production of the isoprenol in accordance with the methods of the invention. Suitable eukaryotic host cells include, but are not limited to, fungal cells. Suitable fungal cells are yeast cells, such as yeast cells of the Saccharomyces genus. Generally, although not necessarily, the host cell is a yeast or a bacterium. Any prokaryotic or eukaryotic host cell may be used in the present method so long as it remains viable after being transformed with a sequence of nucleic acids. In some embodiments, the host cell is not adversely affected by the transduction of the necessary nucleic acid sequences, the subsequent expression of the proteins (i.e., enzymes), or the resulting intermediates required for carrying out the steps associated with the mevalonate pathway. For example, it is preferred that minimal “cross-talk” (i.e., interference) occur between the host cell's own metabolic processes and those processes involved with the mevalonate pathway.

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

The enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments, the host cell is Rhodosporidium toruloides or Pseudomonas putida. In some embodiments, the host cell is a Gram negative bacterium. In some embodiments, the host cell is of the phylum Proteobactera. In some embodiments, the host cell is of the class Gammaproteobacteria. In some embodiments, the host cell is of the order Enterobacteriales. In some embodiments, the host cell is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes.

Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtherias, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

### Recovering Isoprenol Via Gas Strippinc

In some embodiments, the step (b), recovering isoprenol via gas stripping, comprises using the device or apparatus for gas stripping of the present invention. In some embodiments, the step (b), recovering isoprenol via gas stripping, comprises (i) collecting the off-gas isoprenol produced from the culture medium, (ii) passing the off-gas isoprenol through a solvent such that the isoprenol is retained in the solvent, wherein the solvent is an organic solvent or water, and (iii) optionally when the solvent is an organic solvent, stripping the isoprenol in the organic solvent with water, such that the isoprenol strips from the organic solvent into the water.

Gas stripping is a physical separation process where one or more components are removed from a liquid stream by a vapor stream, or vice versa.

Off-gas isoprenol is captured in the solvent, which is then stripped to water to form an aqueous solution comprising isoprenol. Alternately, the off-gas isoprenol is captured directly in water to form an aqueous solution comprising isoprenol. In some embodiments, the solvent or water is chilled, or has a lower temperature. In some embodiments, the lower temperature is lower than room temperature or 25° C., or lower than 20° C., 15° C., 10° C., 5° C., or 4° C. The aqueous solution comprising isoprenol. If stripped directly to water, the isoprenol in the aqueous solution can then be dehydrated into isoprene using the step(s) taught herein. Isoprene can then be recovered as a volatile gas, and the remaining water can then recycled or returned to the gas stripping system. Alternatively, the aqueous isoprenol solution can be concentrated via distillation or membrane distillation prior to dehydration to isoprene. If isoprenol is captured from off-gas in an organic solvent, prior to dehydration it may be converted to an aqueous isoprenol solution via distillation, membrane distillation, or liquid-liquid extraction. In some embodiments, the passing through is bubbling.

Continuous volatilization of isoprenol to fermentor off-gas, coupled with high efficiency capture via off-gas stripping in chilled water or solvent, can yield titers equal to or more than about 20 g/L, the highest titers for isoprenol production to date, and double what has been achieved with in situ extractive fermentation due to product toxicity issues. In some embodiments, the titer is equal to or more than about 10 g/L, 20 g/L, 30 g/L, 40 g/L, 50 g/L, 60 g/L, 70 g/L, 80 g/L, 90 g/L, or 100 g/L. In a particular embodiment, off-gas capture efficiencies in chilled octanol exceeded 90%.

In a particular embodiment, the recovering results in obtaining a mixture of about 97.1% octanol, about 2.6% isoprenol, and about 0.3% isoprenyl acetate is fractionally distilled under reduced pressure. The isoprenol fraction is concentrated and the resulting isoprenol fraction is about 61.7% isoprenol, about 11.7% isoprenyl acetate, and about 26.6% octanol. In some embodiments, the isoprenol recovered has a concentration of equal to or more than about 10%, 20%, 30%, 40%, 50%, 60%, 70%, 80%, or 90% isoprenol. This isoprenol fraction can then be used for subsequent dehydrating the isoprenol into isoprene. This isoprenol fraction can further comprise isoprenyl acetate, and/or the solvent, such as an organic solvent, such as an aliphatic alcohol, such as octanol. Without further purification, this isoprenol fraction having a concentration of equal to or more than about 10%, 20%, 30%, 40%, 50%, 60%, 70%, 80%, or 90% isoprenol can then be used for the subsequent dehydrating the isoprenol into isoprene. In some embodiments, the yield recovery of the isoprenol recovered in the recovering step from the isoprenol produced in the producing step is equal to or more than about 10%, 20%, 30%, 40%, 50%, 60%, 70%, 80%, or 90% recovery.

The present invention provides for a device or apparatus for gas stripping. The present invention provides for a device or apparatus comprising: (a) a first vessel; (b) a second vessel; (c) a first channel in fluid communication between an oxygen/air source and the first vessel, (d) a second channel in fluid communication between the first vessel and the second vessel; and (e) a second channel in fluid communication between the second vessel and the outside of the second vessel; wherein the first vessel contains a culture medium comprising a host cell that produces isoprenol and/or isoprene, and the second vessel contains an organic solvent or water/aqueous solution.

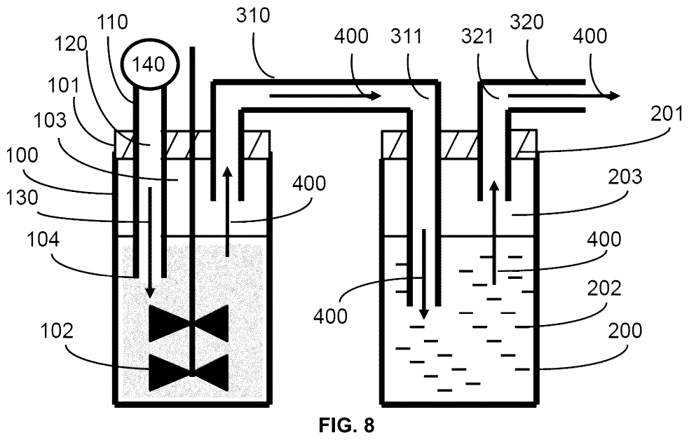

In some embodiments, the device or apparatus comprises the structure shown in FIG. 3 or FIG. 8. 100 is a first vessel. 101 is an optional seal for the 100 first vessel. 102 is a culture medium. 103 is a space filled with gas enclosed within the 100 first vessel. 104 is a means for stirring or agitating the 102 culture medium. 110 is a first channel in fluid communication with the 130 oxygen/air source and 100 first vessel.

110 is a first fluid communication device comprising a 120 first channel which is in fluid communication with the 140 oxygen/air source and the 102 culture medium in the 100 first vessel. 130 is the direction of flow of oxygen/air gas from the 140 oxygen/air source into the 102 culture medium in the 100 first vessel during the operation of the device or apparatus. The oxygen/air flowing into the 102 culture medium provides aeration or an oxygen supply to the host cells in the 102 culture medium. 200 is a second vessel. 201 is an optional seal for the 200 second vessel. 202 is an organic solvent or water/aqueous solution. 203 is a space filled with gas enclosed within the 200 second vessel. 310 is a second fluid communication device comprising a 311 channel which is in fluid communication with the 103 space filled with gas in the 100 first vessel and the 202 organic solvent or water/aqueous solution in the 200 second vessel. 320 is a third fluid communication device comprising a 321 channel which is in fluid communication with the 203 space filled with gas in the 200 second vessel and the outside of the 200 second vessel. 400 is the direction of flow of isoprenol and/or isoprene through the device or apparatus during the operation of the device or apparatus. In some embodiments, the 200 second vessel and/or 202 an organic solvent or water/aqueous solution is cooled so that it has a temperature lower than that of the rest of the device or apparatus. In some embodiments, the lower temperature is lower than room temperature or 25° C., or lower than 20° C., 15° C., 10° C., 5° C., or 4° C.

In some embodiments, the culture medium is a fermentation medium. During the operation of the device or apparatus, the culture medium comprises a host cell capable of producing isoprenol and/or isoprene, and the host cell is producing isoprenol and/or isoprene.

During the operation of the device or apparatus, gaseous molecules produced by the host cell enter the 103 space of the 100 first vessel, and then enter the 310 first channel and travel into the 202 organic solvent or water/aqueous solution in the 200 second vessel.

The solvent is an organic solvent or water. In some embodiments, the organic solvent is an aliphatic alcohol. In some embodiments, the aliphatic alcohol is a straight chain or branched aliphatic alcohol. In some embodiments, the aliphatic alcohol is saturated or unsaturated. In some embodiments, the aliphatic alcohol comprises 1, 2, 3, 4, 5, 6, 7, 8, 9, 10, 11, 12, 13, 14, 15, 16, 17, 18, 19, or 20, or 21 to 34 carbon atoms. In some embodiments, the aliphatic alcohol comprises 4, 5, or 6 to 22, 23, 24, 25, or 26 carbon atoms. In some embodiments, aliphatic alcohol is a straight chain aliphatic saturated or unsaturated aliphatic alcohol. In some embodiments, the aliphatic alcohol comprises one, two, three, four, or five hydroxyl group. In some embodiments, the saturated straight chain aliphatic alcohol is methanol, ethanol, propanol, butanol, pentanol, hexanol, heptanol, octanol, nonanol, or decanol. In some embodiments, the unsaturated straight chain aliphatic alcohol is oleyl alcohol (1-octadecenol) or erucyl alcohol (cis-13-docosen-1-01). In some embodiments, the unsaturated straight chain aliphatic alcohol comprises one, two, three, four, or five C—C double bond. In some embodiments, the saturated branched chain aliphatic alcohol is 2-ethyl-1,3-hexanediol. In some embodiments, the solvent is an aqueous solution or water.

In some embodiments, the aeration used to supply oxygen to the bioreactor is leveraged to strip isoprenol to the fermentor off-gas, ensuring aqueous concentrations below the toxicity threshold. As compared to isoprene, the higher boiling point of isoprenol enables a relatively facile recovery via stripping to either a chilled organic solvent or chilled water. To avoid saturation, this capture medium can be regenerated by removing the isoprene. Rather than typical recovery via distillation, in this system aqueous isoprenol is continuously dehydrated to isoprene, which is then recovered in relatively pure form as a gas. The resulting purified water is then recycled to the off-gas capture system, as either a direct capture medium, or as a stripping solvent for the capture medium in liquid-liquid extraction.

This system avoids the major challenges associated with both isoprenol and isoprene fermentation. Low isoprenol concentrations are maintained in the first vessel, or fermentor, existing aeration is used for in situ product removal, the system can be operated below flammability thresholds, and high purity isoprene is produced with no energy-intensive distillation steps.

Dehydrating Isoprenol into Isoprene

In some embodiments, the step (c) dehydrating isoprenol into isoprene comprises: (i) heating an isoprenol fraction to a temperature equal to or more than about 70° C., 75° C., 80° C., 85° C., 90° C., 95° C., or 100° C., or to a temperature within a range between any two of the preceding temperatures, wherein isoprenol is converted into isoprene, wherein at least some of the isoprene is in a gaseous state; and (ii) condensing or distilling the isoprene in the gaseous state isoprenol. In some embodiments, the (c) dehydrating step comprises using a distillation column, and an overhead temperature of the distillation column is decreased from about 50° C., 45° C., 40° C., 35° C., 34° C., 33° C., or 30° C., to about 25° C., 23° C., 22° C., 21° C., 20° C., 15° C., or 10° C., wherein the isoprene in the gaseous state condenses or distills to form a condensate or distillate. In some embodiments, a condensate or distillate obtained from the condensing or distilling step comprises: equal to or more than about 50%, 55%, 60%, 65%, 70%, 75%, 80%, 85%, 90%, or 95% isoprene, less than about 25%, 20%, 15%, 10%, or 5% of isoprenol isomers and isoprenyl acetate isomers, and less than about 1% of dimers. In some embodiments, the distillation column is first charged with an aqueous solution of salt and acid (such as an inorganic acid, such as HCl, H₂SO₄, or HNO₃). Alternatively, instead of mineral acid or inorganic acid, a recyclable heterogeneous, water stable, acid catalyst is used. In some embodiments, the salt, acid, and water in the aqueous solution have a weight or molar ratio of about 1:0.1:1.5. In some embodiments, the salt is any metal halide, sulfate, or nitrate. In some embodiments, the metal is any alkali, alkaline earth, or transition metal. In some embodiments, the aqueous solution of salt and acid comprises sodium chloride, HCl, and water. (6.01 g). In some embodiments, the aqueous solution is agitated or stirred such as agitated or stirred vigorously. In some embodiments, the agitation or stirring of the aqueous solution takes place at a temperature equal to or more than about 95° C., 90° C., 85° C., 80° C., 75° C., 70° C., 65° C., or 60° C.

The isoprenol produced is converted into isoprene by a dehydration reaction:

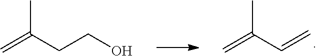

The isoprene formed evaporates from the solution. In some embodiments, the isoprenol in the solution is reduced, or is entirely converted into isoprene, such that the solution is free of isoprenol, or essentially free of isoprenol. In both cases the isoprenol-free water could be returned as a recovery or extraction solvent. In some embodiments, the isoprene is distilled from the metal, degassed and placed under a nitrogen atmosphere. In some embodiments, the aqueous dehydration reaction mixture generates one or more minor byproducts, such as cyclic isoprene dimer.

Such minor byproducts, such as cyclic isoprene dimer, can be readily isolated from the aqueous dehydration reaction mixture and combined with the DMCOD.

Isoprenol is converted into isoprene through the catalytic dehydration of isoprenol. The biologically produced isoprenol is distilled from the capture solvent and redissolved in water to execute the dehydration, future development would be necessary to demonstrate continuous dehydration and regeneration of the capture solvent in a directly couple system. An advantage of the present invention is that isoprene can volatilize directly from dehydration into an oxygen-free environment, thereby eliminating flammability risk.

Converting Isoprene into 1,4-Dimethylcyclooctane (DMCO)

In some embodiments, the step (d) converting isoprene into DMCO comprises: (i) [4+4]-cyclodimerizing of the isoprene into a 1,6-dimethyl-1,5-cyclooctadiene (DMCOD), and (ii) hydrogenating the DMCOD into a DMCO. In some embodiments, the [4+4]-cyclodimerizing of the isoprene into DMCOD comprises contacting isoprene with a metal catalyst such that the isoprene is converted into DMCOD. In some embodiments, the hydrogenating the DMCOD comprises contacting DMCOD with a metal catalyst such that the DMCOD is converted into DMCO. In some embodiments, the metal catalyst comprises a transition metal, such as Fe, Pt, Raney Ni and Pd/C, such as 10% Pd/C. In some embodiments, the metal catalyst is platinum (IV) oxide.

The one or more C₁₀H₁₄ aromatic compounds can be blended with either jet fuel or diesel fuel. In some embodiments, the method further comprises: mixing DMCO with a fuel additive to produce a fuel mixture. In some embodiments, the method further comprises: mixing any one, two, three, four, five, six, or all of DMCO, cyclic isoprene dimer, DMCOD, one or more C₁₀H₁₄ aromatic compounds, hydrogenated C15 oligomer, hydrogenated C15 oligomer, with a jet fuel or diesel fuel, and/or with a fuel additive to produce a fuel mixture. In some embodiments, the fuel additive includes one of antioxidants, thermal stability improvers, cetane improvers, stabilizers, cold flow improvers, combustion improvers, anti-foams, anti-haze additives, corrosion inhibitors, lubricity improvers, icing inhibitors, injector cleanliness additives, smoke suppressants, drag reducing additives, metal deactivators, dispersants, detergents, demulsifiers, dyes, markers, static dissipaters, biocides, and combinations thereof.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Capture and Conversion of isoprenol (3-methylbut-3-en-1-ol) into isoprene

The production of isoprene has the challenges of having high toxicity, poor liquid-liquid partitioning, and high volatility of isoprene. The liquid-liquid extraction fermentation of isoprenol (FIG. 2) is less efficient than the off-gas stripping method of the present invention (FIG. 3).

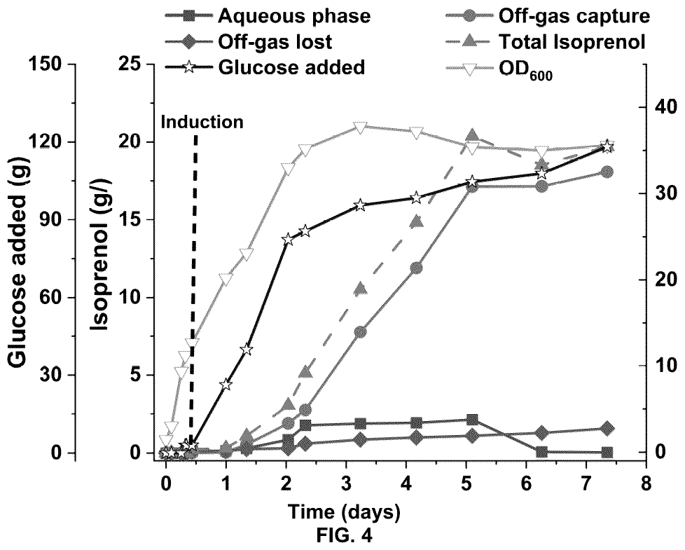

Gas stripping can result in a 20 g/L isoprenol titer (FIG. 4). This method overcomes the toxicity challenge. It also has a lower cost recovery. The gas stripping makes use of the existing aeration, and has an over 90% recovery of the isoprenol.

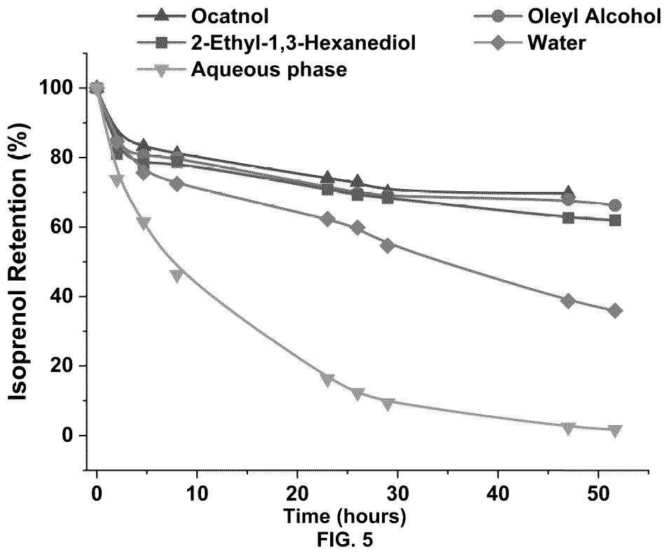

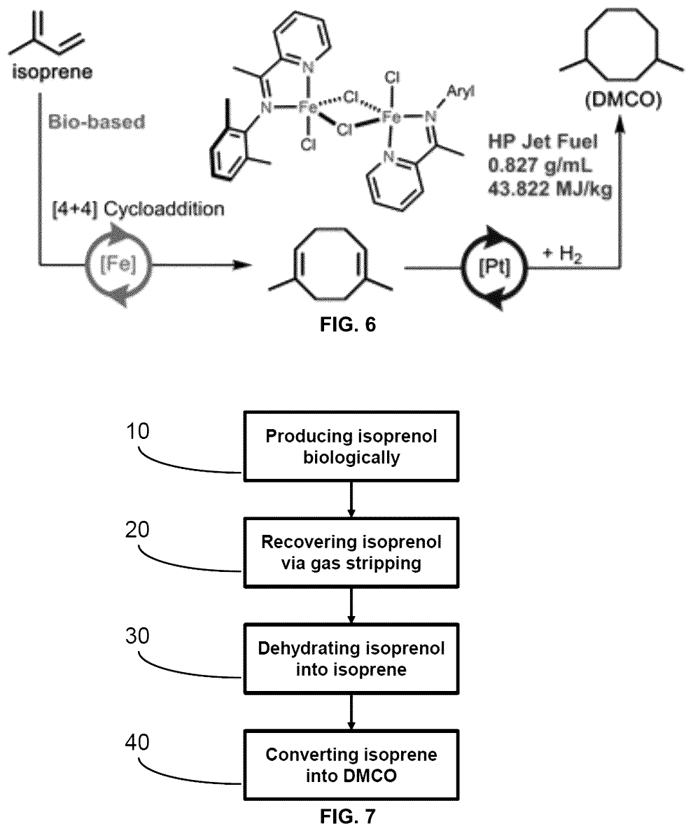

Multiple solvents are effective for the capture of isoprenol (as shown in FIG. 5). Even chilled water is relatively effective for capture pre-saturation. The water after use can be recycled back into the stripping process. The isoprenol is captured in chilled solvent, and then stripped to water. Alternately, the isoprenol is captured directly in chilled water. The isoprenol in the aqueous solution can then be catalytically dehydrated into pure isoprene. The pure water can then be returned to the gas stripping system.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A method for producing isoprene, the method comprising: (a) producing isoprenol (3-methyl-3-buten-1-ol) biologically; and, (b) recovering the isoprenol via gas stripping.

2. The method of claim 1, wherein the producing step comprises: (i) providing for a host cell capable of producing isoprenol, and (ii) culturing the host cell under a condition wherein isoprenol is produced.

3. The method of claim 2, wherein the gas stripping comprises aeration of oxygen into a culture comprising the host cell producing isoprenol.

4. The method of claim 2, wherein the host cell is a genetically modified host cell capable of producing more isoprenol than the unmodified host cell.

5. The method of claim 2, wherein the host cell comprises (a) an increased expression of phosphomevalonate decarboxylase (PMD), or a homologous enzyme thereof, (b) an increased expression of a phosphatase, or a homologous enzyme thereof, capable of converting isopentenol into isoprenol, and (c) optionally the genetically modified host cell does not express, or has a decreased expression of one or more of NudB, PMK, and/or PMD, or a homologous enzyme thereof.

6. The method of claim 5, wherein the PMD comprises a polypeptide sequence having at least 70% identity with SEQ ID NO:1 or 2; and comprising (a) E at position 73, S at position 108, N at position 110, A at position 119, S at position 120, S at position 121, A at position 122, S at position 155, R at position 158, S at position 208, and/or D at position 302 corresponding to SEQ ID NO:1; or (b) Eat position 71, S at position 94, N at position 96, A at position 105, S at position 106, S at position 107, A at position 108, S at position 141, R at position 144, S at position 192, and/or D at position 283 corresponding to SEQ ID NO:2.

7. The method of claim 1, wherein the gas stripping comprises (i) collecting off-gas isoprenol produced, (ii) passing the off-gas isoprenol through a solvent such that the isoprenol is retained in the solvent, wherein the solvent is an organic solvent or water, and (iii) optionally when the solvent is an organic solvent, stripping the isoprenol in the organic solvent with water, such that the isoprenol strips from the organic solvent into the water.

8. The method of claim 1, wherein the producing and gas stripping steps takes place via a device or apparatus comprising: (a) a first vessel; (b) a second vessel; (c) a first channel in fluid communication between an oxygen/air source and the first vessel, (d) a second channel in fluid communication between the first vessel and the second vessel; and (e) a second channel in fluid communication between the second vessel and the outside of the second vessel; wherein the first vessel contains a culture medium comprising a host cell that produces isoprenol, and the second vessel contains an organic solvent or water/aqueous solution.

9. The method of claim 7, wherein the gas stripping comprises gas stripping into an organic solvent, or an aqueous phase.

10. The method of claim 8, wherein the gas stripping comprises gas stripping into an organic solvent, and the organic solvent is an aliphatic alcohol.

11. The method of claim 8, wherein the gas stripping comprises gas stripping into an aqueous phase.

12. The method of claim 8, wherein the organic solvent, or the aqueous phase, has a temperature from 4° C. to 25° C.

13. The method of claim 1, the method comprising: (c) dehydrating the isoprenol into isoprene.

14. The method of claim 13, wherein the dehydrating step comprises: (i) heating an isoprenol fraction to a temperature equal to or more than about 70° C. to 100° C., wherein isoprenol is converted into isoprene, wherein at least some of the isoprene is in a gaseous state; and (ii) condensing or distilling the isoprene in the gaseous state isoprenol.

15. The method of claim 13, wherein the dehydrating step comprises: using a distillation column, and an overhead temperature of the distillation column is decreased from about 50° C. to 30° C., to about 10° C. to 25° C., wherein the isoprene in the gaseous state condenses or distills to form a condensate or distillate.

16. The method of claim 13, the method comprising: (d) converting the isoprene into 1,4-dimethylcyclooctane (DMCO).

17. The method of claim 16, wherein the converting step comprises: (i) [4+4]-cyclodimerizing of the isoprene into a 1,6-dimethyl-1,5-cyclooctadiene (DMCOD), and (ii) hydrogenating the DMCOD into a DMCO.

18. The method of claim 17, wherein the [4+4]-cyclodimerizing step comprises contacting isoprene with a metal catalyst such that the isoprene is converted into DMCOD.

19. The method of claim 17, wherein the hydrogenating step comprises contacting DMCOD with a metal catalyst such that the DMCOD is converted into DMCO.

20. The method of claim 17, wherein metal catalyst comprises a Fe, Pt, Raney Ni, Pd/C, or platinum (IV) oxide.

21. The method of claim 16, the method comprising: mixing DMCO with a fuel additive to produce a fuel mixture.


SEQUENCE LISTING (2 sequences)

SEQ ID NO: 1 - protein, 396 aa, Saccharomyces cerevisiae
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 2 - protein, 327 aa, Saccharomyces epidermis
MVKSGKARAHTNIALIKYWGKADETYIIPMNNSLSVTLDRFYTETKVTFDPDFTEDCLILNGNEVNAKEKEKIQNYMNIVRDLAGNRLHARIESENYVPTAAGLASSASAYAALAAACNEALSLNLSDTDLSRLARRGSGSASRSIFGGFAEWEKGHDDLTSYAHGINSNGWEKDLSMIFVVINNQSKKVSSRSGMSLTRDTSRFYQYWLDHVDEDLNEAKEAVKNQDFQRLGEVIEANGLRMHATNLGAQPPFTYLVQESYDAMAIVEQCRKANLPCYFTMDAGPNVKVLVEKKNKQAVMEQFLKVFDESKIIASDIISSGVEIIK